(19) United States
(12) Patent Application Publication
(10) Pub. No.: US 2022/0079887 A1
(43) Pub. Date: Mar. 17, 2022

(54) TRANSDERMAL THERAPEUTIC SYSTEM WITH DIFFUSION BARRIER

(71) Applicant: LTS Lohmann Therapie-Systeme AG, Andernach (DE)

(72) Inventors: Nico REUM, Mending (DE); Marco EMGENBROICH, Rheinbach (DE); Sandra WIEDERSBERG, Steigra (DE)

(21) Appl. No.: 17/298,551

(22) PCT Filed: Nov. 25, 2019
(86) PCT No.: PCT/EP2019/082448

§ 371 Date: May 29, 2021

(30) Foreign Application Priority Data
Nov. 30, 2018 (DE) 10 2018 130 469.2

(51) Int. Cl. A61K 9/70; A61K 45/06

(52) U.S. Cl. CPC A61K 9/7084; A61K 45/06

(57) ABSTRACT

The present invention relates to a transdermal therapeutic system (1) comprising
an active-substance carrier layer (2) having at least one active-substance-containing polymer matrix (3) applied to the active-substance carrier layer (2), said matrix comprising at least one pressure-sensitive adhesive and at least one pharmacologically active substance absorbable through human or animal skin, and
an adhesive carrier layer (6) coated as completely as possible with an active-substance-free pressure-sensitive adhesive (7), said adhesive carrier layer being bonded by means of the active-substance-free pressure-sensitive adhesive (7) directly to the flat side of the active-substance carrier layer (2) facing away from the active-substance-containing polymer matrix (3),
   wherein the adhesive carrier layer (6) projecting circumferentially beyond the edge of the active-substance carrier layer (2), wherein the system is characterized in that between the active-substance-containing polymer matrix (3) and the active-substance-free pressure-sensitive adhesive (7) there is a circumferential zone (4) having a reduced thickness of coating with the active-substance-containing polymer matrix (3) and/or with the active-substance-free pressure-sensitive adhesive (7). The invention further relates to the use of this transdermal therapeutic system and to a kit that contains it.

The present invention relates to a transdermal therapeutic system (abbreviated “TTS”) comprising an active-substance carrier layer having at least one active-substance-containing polymer matrix applied to the active-substance carrier layer, said matrix comprising at least one pressure-sensitive adhesive and at least one pharmacologically active substance absorbable through human or animal skin, and also an adhesive carrier layer coated as completely as possible with an active-substance-free pressure-sensitive adhesive, said adhesive carrier layer being bonded by means of the active-substance-free pressure-sensitive adhesive directly to the flat side of the active-substance carrier layer facing away from the active-substance-containing polymer matrix, the adhesive carrier layer projecting circumferentially beyond the edge of the active-substance carrier layer. The invention further relates to the use of this transdermal therapeutic system and to a kit that contains it.

Transdermal therapeutic systems (TTS) are planar pharmaceutical products with a layered structure, in which one or more active substances with or without excipients (e.g. penetration accelerators) are embedded in an optionally pressure-sensitive-adhesive polymer matrix. This polymer matrix is usually produced by coating a backing film with the active-substance-containing polymer composition and then furnishing it with a cover film that remains on the skin even during application of the transdermal therapeutic system. The backing film serves as a protective layer for the polymer matrix while undergoing storage and optionally as an application aid for the later use of the transdermal therapeutic system.

Transdermal therapeutic systems enable a continuous supply of active substance over the entire application period. In their concentration-time profiles, they are therefore comparable to continuous-drip infusions. Numerous transdermal therapeutic systems with different active substances and active substance combinations are nowadays on the pharmaceutical market. One of the most important indication areas for transdermal therapeutic systems is hormone replacement therapy, especially in menopausal women. In the early years of transdermal hormone replacement therapy, it was mainly estrogen-containing single-agent products that were used for this purpose. More recently, however, transdermal therapeutic systems have become available that comprise a combination of estrogens (e.g. 17β-estradiol) and gestagens (e.g. norethisterone). Testosterone, the male sex hormone, is another of the group of steroid hormones used in hormone replacement therapy, especially in the treatment of hypogonadism.

A number of commercially available transdermal therapeutic systems are constructed as so-called matrix systems. These are systems in which the active substance is present in dissolved or suspended form in a polymer matrix furnished with a pressure-sensitive adhesive or non-pressure-sensitive adhesive. Such polymer matrices consist mostly of pressure-sensitive adhesives based on polyacrylates.

Since the pharmacologically active substances contained in the active-substance patch of the transdermal therapeutic system are in many applications costly, the active-substance patches are often cut cornered, for example square, in order to minimize cutting losses. Since cornered patches detach from the skin more easily, the fixing patch, which usually has rounded corners or is round overall, is affixed over it for a better hold. However, such arrangements give rise to undesirable migration phenomena of the active substance and/or of one or more excipients into the directly adjacent pressure-sensitive-adhesive matrix of the fixing patch. Because the active substance may no longer be able to migrate from the pressure-sensitive-adhesive matrix into the skin, this can lead to uncontrolled losses of active substance and consequent failure to achieve the desired dosing of the active substance. Various approaches to counter this migration problem are known from the prior art.

US 2017/0290779 A1 discloses a patch for dermal application, the patch comprising an active reservoir layer, a carrier layer projecting beyond this layer, and a further carrier layer projecting beyond the first two layers that is coated with an adhesive. The reservoir layer may comprise a pressure-sensitive adhesive and/or a non-adhesive polymer matrix. The production of this layer structure is extremely complex and necessitates a high degree of manufacturing precision in its composition.

WO 2016/081616 A2 describes a patch formulation for the transdermal delivery of water-soluble active substances, peptides, proteins, and oligosaccharides. The patch comprises a layer comprising an active substance and a polymer matrix, and an adhesive layer, the adhesive layer projecting beyond the active substance/polymer layer. The adhesive layer additionally comprises an adhesive-free zone that projects beyond the active substance/polymer layer.

EP 0 755 284 B1 teaches a topical dressing for dermal and/or transdermal administration of a substance, the dressing comprising a carrier layer coated with an adhesive. The carrier layer additionally comprises a circular cut-out area that is free of adhesive. In the cut-out area, a recess is defined into which an active-substance-containing pad is inserted, the pad having a smaller diameter than the cut-out area. This gives rise to an intermediate zone of reduced layer thickness between the active-substance-containing pad and the adhesive layer.

In the case of the two abovementioned solutions, it can be perceived as disadvantageous that the coating of a carrier layer with different coating materials and coating structures at different points during production is complex and prone to errors. Moreover, in the case of this construction, the carrier layer must be compatible with all coating materials, which limits the choice of materials.

With this prior art as a starting point, the object of the present invention was to provide a transdermal therapeutic system of the type mentioned in the introduction that can be produced more inexpensively and that at the same time has good adhesive properties on skin and in which migration of the pharmacologically active substance into the adhesive of the fixing patch is largely prevented.

This object is achieved in a transdermal therapeutic system of the type mentioned in the introduction by providing, between the active-substance-containing polymer matrix and the active-substance-free pressure-sensitive adhesive of the adhesive carrier layer, a zone having a reduced thickness of coating with the active-substance-containing polymer matrix and/or with the active-substance-free pressure-sensitive adhesive, said zone acting as a diffusion barrier that prevents lateral diffusion of the pharmacologically active substance and/or of the one or more excipients into the adhesive of the adhesive carrier layer.

The invention thus relates to a transdermal therapeutic system comprising
an active-substance carrier layer having at least one active-substance-containing polymer matrix applied to the active-substance carrier layer, said matrix comprising at least one pressure-sensitive adhesive and at least one pharmacologically active substance absorbable through human or animal skin, and
an adhesive carrier layer coated as completely as possible with an active-substance-free pressure-sensitive adhesive, said adhesive carrier layer being bonded by means of the active-substance-free pressure-sensitive adhesive directly to the flat side of the active-substance carrier layer facing away from the active-substance-containing polymer matrix,

the adhesive carrier layer projecting circumferentially beyond the edge of the active-substance carrier layer, the system being characterized in that between the active-substance-containing polymer matrix and the active-substance-free pressure-sensitive adhesive there is a circumferential zone having a reduced thickness of coating with the active-substance-containing polymer matrix and/or with the active-substance-free pressure-sensitive adhesive.

The circumferential edge zone having a reduced thickness of coating is preferably produced by guiding a punching tool onto the active-substance carrier layer that is initially coated over its entire surface with active-substance-containing polymer matrix, said tool comprising a hollow cylinder and a cutting blade arranged to the outside of the hollow cylinder, more particularly concentrically and directly adjoining it. In this process, the hollow cylinder is first pressed onto the coated active-substance carrier layer. This causes a lateral displacement of the adhesive that results in a substantial reduction in the thickness of coating with the active-substance-containing polymer matrix in this zone. The cutting blade is then used to punch out the active-substance carrier layer with a circumferential edge zone having a reduced thickness of coating with the active-substance-containing polymer matrix. This procedure is particularly advantageous, because in this process it is possible to start from a carrier material coated over its entire surface with an active-substance-containing polymer matrix as the active-substance carrier layer, allowing the coated active-substance carrier layer to be produced in a single operation without loss of material and without complex local coating methods. Even if a residual layer thickness of active-substance-containing polymer matrix remains during this process, this is nevertheless sufficient to almost completely prevent diffusion of the pharmacologically active substance into the adhesive of the adhesive carrier layer.

The present invention thus also provides a process for producing a transdermal therapeutic system of the invention, comprising the steps of
providing an active-substance carrier layer having at least one active-substance-containing polymer matrix applied to the active-substance carrier layer, said matrix comprising at least one pressure-sensitive adhesive and at least one pharmacologically active substance absorbable through human or animal skin, and
bonding an adhesive carrier layer coated as completely as possible with an active-substance-free pressure-sensitive adhesive by means of the active-substance-free pressure-sensitive adhesive directly to the flat side of the active-substance carrier layer facing away from the active-substance-containing polymer matrix,

the adhesive carrier layer projecting circumferentially beyond the edge of the active-substance carrier layer, the process being characterized in that between the active-substance-containing polymer matrix and the active-substance-free pressure-sensitive adhesive there is a circumferential zone having a reduced thickness of coating with the active-substance-containing polymer matrix and/or with the active-substance-free pressure-sensitive adhesive, the circumferential edge zone with a reduced thickness of coating preferably being produced by guiding a punching tool onto the material for the active-substance carrier layer that is coated over its entire surface with active-substance-containing polymer matrix, said tool comprising a hollow cylinder and a cutting blade arranged to the outside of the hollow cylinder, and more particularly concentrically and directly adjoining it, the hollow cylinder being first pressed onto the coated active-substance carrier layer, causing a reduction in the thickness of coating with the active-substance-containing polymer matrix, after which the cutting blade is used to punch out the active-substance carrier layer with a circumferential edge zone having a reduced thickness of coating with the active-substance-containing polymer matrix. The cutting blade then preferably moves initially to its starting position and only then is the hollow cylinder raised. The punching is preferably executed from the flat side facing away from the active-substance-containing polymer matrix, although it is also possible to punch from the side of the active-substance-containing polymer matrix.

For final assembly of the transdermal therapeutic system, the punched blank obtained as described above is bonded by its flat side facing away from the active-substance-containing polymer matrix to the adhesive side of the adhesive carrier layer such that the adhesive carrier layer projects circumferentially beyond the edge of the active-substance carrier layer. The transdermal therapeutic system thus produced is then typically further covered on the adhesive side with a protective layer in the form of a release liner and packaged.

The invention further relates to the use of a transdermal therapeutic system of the invention for the treatment of hypogonadism, for hormone replacement therapy, of Alzheimer's, of Parkinson's, of multiple sclerosis, of bipolar disorders, of muscle tension, of severe pain, of high blood pressure or for contraception, to name just a few.

Another object of the present invention relates to a kit comprising a transdermal therapeutic system of the invention in an outer packaging and optionally also an instructions for use containing directions for use in accordance with the invention.

For the active-substance-containing polymer matrix and the active-substance-free pressure-sensitive adhesive, it is possible to independently use pressure-sensitive-adhesive systems known to those skilled in the art that are suitable for use on the skin. A pressure-sensitive adhesive for the purposes of the present invention is an adhesive that is permanently sticky within a range from 0 to 40° C., in particular at room temperature, and forms good adhesion to different surfaces under slight pressure, in particular to human skin. This is also referred to as “tacky”. Suitable adhesives are known to those skilled in the art from the technical field of adhesive tapes, in particular medical adhesive tapes or first-aid dressings. Suitable pressure-sensitive adhesives have for example a glass transition temperature T_{g} of ≤−10° C. The glass transition temperature T_{g} can be determined by DSC (differential scanning calorimetry) using a Mettler DSC 12E (Mettler Toledo GmbH, Giessen, Germany) at a heating rate of 10 K/min. Examples of pressure-sensitive adhesives that can be used are those disclosed in DE 101 41 652 A1. Suitable pressure-sensitive adhesives are commercially available for example under the brand names Duro-Tak® and Gelva® (both from Henkel AG & Co. KGaA). Suitable silicone adhesives (BIO-PSA 7-4101, BIO-PSA 7-4102, BIO-PSA 7-4201, BIO-PSA 7-4202, BIO-PSA 7-4301, BIO-PSA 7-4302, BIO-PSA 7-4401, BIO-PSA 7-4402, BIO-PSA 7-4501, BIO-PSA 7-4502, BIO-PSA 7-4601, BIO-PSA 7-4602), silicone-acrylate hybrid systems (7-6101 SilAc Hybrid PSA, 7-6102 SilAc Hybrid PSA, 7-6301 SilAc Hybrid PSA, 7-6302 SilAc Hybrid PSA), and 2-component silicone adhesive (MG7-9700 kit (A&B), MG7-9800 kit (A&B), MG7-9850 Kit (A&B), MG7-9900 kit (A&B), MG7-1010 kit (A&B)) are commercially available from Dow Corning, polyisobutylenes (Oppanol B10 N, Oppanol B10 SFN, Oppanol B11 SFN, Oppanol B12 N, Oppanol B12 SFN, Oppanol B13 SFN, Oppanol B14 SFN, Oppanol B15 N, Oppanol B15 SFN, Oppanol N50, Oppanol N50 SF, Oppanol N80, Oppanol N100, Oppanol N150) from BASF SE, styrene-isoprene-styrene block copolymers (JSR SIS 5505, JSR SIS 5403, JSR SIS 5250, JSR SIS 5229, JSR SIS 5002) from JSR Life Science in Japan, and suitable polyurethane pressure-sensitive adhesives (Transform™ TPU) from Lubrizol.

The transdermal therapeutic system of the invention comprises an active-substance carrier layer and an adhesive carrier layer. The carrier material used for the active-substance carrier layer and the adhesive carrier layer can be the same or different. The carrier can be in the form of a film, woven fabric, laid fabric, nonwoven fabric or knitted fabric. The carrier is expediently flexible enough that the system is able to fit to the contours of the skin. Suitable carrier materials include conventional flexible carrier materials used for pressure-sensitive adhesive tapes, such as polyethylene, especially low-density polyethylene, linear low-density polyethylene, metallocene polyethylene, high-density polyethylene, polypropylene, polyesters such as polyethylene terephthalate, statistically oriented nylon fibers, ethylene-vinyl acetate copolymer, polyurethane, natural fibers such as rayon and the like. Carriers that are layered, such as polyethylene terephthalate-aluminum-polyethylene composites, are also suitable.

The carrier should be essentially inert toward the components of the active-substance-containing polymer matrix and of the active-substance-free pressure-sensitive adhesive. The thickness of the carrier material depends on the desired requirements and is for example within a range from 5 to 100 μm.

The carrier material used is preferably a polyethylene terephthalate film, the thickness of which is further preferably within a range from 10 to 40 μm.

The uncoated side of the active-substance carrier layer and in particular of the adhesive carrier layer can have an outer coating, for example in skin colors, to make the patch less conspicuous when worn.

The surface area of the transdermal therapeutic system depends on the requirements and is typically 1.0 to 250 cm².

The polymer matrix of the active-substance-containing polymer matrix and of the active-substance-free pressure-sensitive adhesive can be provided with a protective layer covering the entire surface, the protective layer on the flat side facing the active-substance-containing polymer matrix in particular being furnished with an adhesion-reducing coating, preferably with siliconization or, particularly in the case of silicone adhesives, with fluorosiliconization. The adhesion-reducing coating permits easier removal of the protective layer, since it is peeled off before application of the TTS so as to expose the pressure-sensitive-adhesive surfaces. Suitable protective layers include conventional separating layers comprising a known sheet material such as polyester sheet, polyethylene sheet, polystyrene sheet, or polyethylene-coated paper coated with a suitable fluoropolymer- or silicone-based coating.

The invention relates in particular to the following embodiments:

In a first embodiment, the invention relates to a transdermal therapeutic system comprising
an active-substance carrier layer having at least one active-substance-containing polymer matrix applied to the active-substance carrier layer, said matrix comprising at least one pressure-sensitive adhesive and at least one pharmacologically active substance absorbable through human or animal skin, and
an adhesive carrier layer coated as completely as possible with an active-substance-free pressure-sensitive adhesive, said adhesive carrier layer being bonded by means of the active-substance-free pressure-sensitive adhesive directly to the flat side of the active-substance carrier layer facing away from the active-substance-containing polymer matrix,

the adhesive carrier layer projecting circumferentially beyond the edge of the active-substance carrier layer, characterized in that between the active-substance-containing polymer matrix and the active-substance-free pressure-sensitive adhesive there is a circumferential zone having a reduced thickness of coating with the active-substance-containing polymer matrix and/or with the active-substance-free pressure-sensitive adhesive.

In a second embodiment, the invention relates to a system according to embodiment 1, characterized in that the adhesive carrier layer is coated as completely as possible with the active-substance-free pressure-sensitive adhesive and is bonded to the active-substance carrier layer, wherein the active-substance carrier layer has a circumferential edge zone having a reduced thickness of coating with the active-substance-containing polymer matrix.

In a third embodiment, the invention relates to a system according to embodiment 2, characterized in that the circumferential edge zone having a reduced thickness of coating is produced or producible by guiding a punching tool onto the material for the active-substance carrier layer that is coated over its entire surface with active-substance-containing polymer matrix, said tool comprising a hollow cylinder and a cutting blade arranged to the outside of the hollow cylinder, more particularly concentrically and directly adjoining it, the hollow cylinder being first pressed onto the coated active-substance carrier layer, causing a reduction in the thickness of coating with the active-substance-containing polymer matrix, after which the cutting blade is used to punch out the active-substance carrier layer with a circumferential edge zone having a reduced thickness of coating with the active-substance-containing polymer matrix.

In a fourth embodiment, the invention relates to a system according to embodiment 1, characterized in that the adhesive carrier layer, where it borders the active-substance carrier layer, has a circumferential zone having a reduced thickness of coating with the active-substance-free pressure-sensitive adhesive.

In a fifth embodiment, the invention relates to a system according to any of the preceding embodiments, characterized in that, in the system, the active-substance-containing polymer matrix is applied directly to the active-substance carrier layer and the active-substance-free pressure-sensitive adhesive directly to the adhesive carrier layer, there being no further layer between the flat side of the active-substance carrier layer facing away from the active-substance-containing polymer matrix and the active-substance-free pressure-sensitive adhesive of the adhesive carrier layer.

In a sixth embodiment, the invention relates to a system according to any of the preceding embodiments, characterized in that the polymer matrix of the active-substance-containing polymer matrix and of the active-substance-free pressure-sensitive adhesive is independently selected from acrylates, silicone pressure-sensitive adhesives, polyisobutylenes, SIS copolymers, silicone-acrylate hybrid systems, for example those marketed by Dow Corning Healthcare Solutions, and mixtures thereof.

In a seventh embodiment, the invention relates to a system according to any of the preceding embodiments as claimed in any of the preceding claims, characterized in that the pharmacologically active substance is selected from the group of α-adrenergic agonists, β-adrenergic agonists, α-adrenergic antagonists, β-adrenergic antagonists, analgesics (narcosis-inducing), analgesics (non-narcosis-inducing), androgens, anesthetics, antiallergics, antiandrogens, antianginals, antiarrhythmics, penicillins, antidiabetics, antidementia drugs, antihistamines, antimigraine drugs, hydrogenated ergot alkaloids, calcium-channel blockers, hormones, serotonin antagonists, platelet-aggregation inhibitors, antidepressants, bronchodilators, estrogens, gestagens, vasodilators and nicotine or mixtures thereof.

In an eighth embodiment, the invention relates to a system according to any of the preceding embodiments, characterized in that the circumferential zone of reduced coating thickness is not more than 20% of the thickness of the polymer matrix of the active-substance-containing polymer matrix and/or of the active-substance-free pressure-sensitive adhesive, in particular not more than 15%, preferably 0.1 to 12%. In the zone of reduced coating thickness, it is also possible for the active-substance-containing polymer matrix and/or the active-substance-free pressure-sensitive adhesive to be completely removed.

In a ninth embodiment, the invention relates to a system according to any of the preceding embodiments, characterized in that the circumferential zone of reduced coating thickness has a width of 0.05 to 5.0 mm, in particular of 0.1 to 3.0 mm.

In a tenth embodiment, the invention relates to a system according to any of the preceding embodiments, characterized in that the circumferential zone of reduced coating thickness is essentially unbroken.

In an eleventh embodiment, the invention relates to a system according to any of the preceding embodiments, characterized in that the coating thickness of the active-substance-containing polymer matrix and/or of the active-substance-free pressure-sensitive adhesive is 20 to 800 μm, in particular 40 to 400 μm.

In a twelfth embodiment, the invention relates to a system according to any of the preceding embodiments, characterized in that a protective layer covering the entire surface of the polymer matrix of the active-substance-containing polymer matrix and of the active-substance-free pressure-sensitive adhesive is provided, wherein the protective layer on the flat side facing the active-substance-containing polymer matrix is equipped in particular with an adhesion-reducing coating, preferably with siliconization or fluorosil-iconization.

In a thirteenth embodiment, the invention relates to a process for producing a transdermal therapeutic system according to any of embodiments 1 to 12, comprising the steps of
providing an active-substance carrier layer having at least one active-substance-containing polymer matrix applied to the active-substance carrier layer, said matrix comprising at least one pressure-sensitive adhesive and at least one pharmacologically active substance absorbable through human or animal skin, and
bonding an adhesive carrier layer coated as completely as possible with an active-substance-free pressure-sensitive adhesive by means of the active-substance-free pressure-sensitive adhesive directly to the flat side of the active-substance carrier layer facing away from the active-substance-containing polymer matrix,
wherein the adhesive carrier layer projecting circumferentially beyond the edge of the active-substance carrier layer,
characterized in that
between the active-substance-containing polymer matrix and the active-substance-free pressure-sensitive adhesive there is a circumferential zone having a reduced thickness of coating with the active-substance-containing polymer matrix and/or with the active-substance-free pressure-sensitive adhesive, wherein the circumferential edge zone with a reduced thickness of coating preferably being produced by guiding a punching tool onto the material for the active-substance carrier layer that is coated over its entire surface with active-substance-containing polymer matrix, said tool comprising a hollow cylinder and a cutting blade arranged to the outside of the hollow cylinder, more particularly concentrically and directly adjoining it, the hollow cylinder being first pressed onto the coated active-substance carrier layer, causing a reduction in the thickness of coating with the active-substance-containing polymer matrix, after which the cutting blade is used to punch out the active-substance carrier layer with a circumferential edge zone having a reduced thickness of coating with the active-substance-containing polymer matrix.

In a fourteenth embodiment, the invention relates to the use of a transdermal therapeutic system according to any of embodiments 1 to 12 for the treatment of the human or animal body, in particular of hypogonadism, for hormone replacement therapy, of Alzheimer's, of Parkinson's, of multiple sclerosis, of bipolar disorders, of muscle tension, of severe pain, of high blood pressure or for contraception.

In a fifteenth embodiment, the invention relates to a kit comprising at least one transdermal therapeutic system according to any of embodiments 1 to 12 in an outer packaging and optionally also an instructions for use containing directions for use in accordance with embodiment 14.

### EXAMPLE(S)

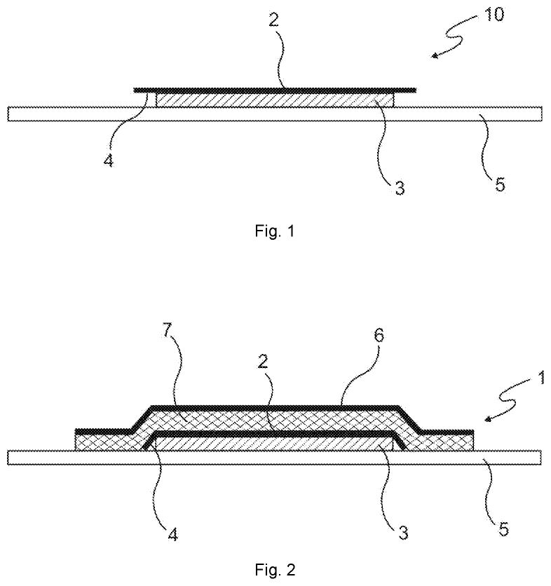

The present invention is elucidated in more detail hereinbelow with reference to the working examples shown in FIGS. 1 and 2. In the figures

FIG. 1 shows an intermediate stage in the production of a transdermal therapeutic system according to the invention, and

FIG. 2 shows an embodiment of the finished transdermal therapeutic system according to the invention as shown in FIG. 2.

FIG. 2 depicts an embodiment of a transdermal therapeutic system 1 according to the invention. The transdermal therapeutic system 1 comprises an active-substance carrier layer 2 having at least one active-substance-containing polymer matrix 3 applied to the active-substance carrier layer 2, said matrix comprising at least one pressure-sensitive adhesive and at least one pharmacologically active substance absorbable through human or animal skin, in the present case scopolamine. The active-substance carrier layer 2 has a circumferential edge zone 4 having a reduced thickness of coating with the active-substance-containing polymer matrix and is applied onto a protective layer 5 that covers the entire surface of the active-substance-containing polymer matrix 3 and projects beyond this at the edge and is provided with a silicone coating to reduce adhesion of the pressure-sensitive adhesive.

On the flat side facing the active-substance-containing polymer matrix 3, the active-substance carrier layer 2 is completely covered by an adhesive carrier layer 6, which is bonded directly to the active-substance carrier layer 2 by means of an active-substance-free pressure-sensitive adhesive 7. The adhesive carrier layer 6 is coated over its entire surface with the active-substance-free pressure-sensitive adhesive 7. The bonding of the adhesive carrier layer 6 to the active-substance carrier layer 2 results in the circumferential zone 4 with a reduced thickness of coating with the active-substance-containing polymer matrix being bent over all the way round. Both the pressure-sensitive adhesive of the active-substance-containing polymer matrix 3 and the active-substance-free pressure-sensitive adhesive 7 are in the present case a silicone-based pressure-sensitive adhesive.

In the production of the transdermal therapeutic system 1 according to the invention, an active-substance carrier layer 2 is first coated with an active-substance-containing polymer matrix 3 comprising at least one pressure-sensitive adhesive, in the present case a silicone-based pressure-sensitive adhesive, and at least one pharmacologically active substance absorbable through human or animal skin, in the present case scopolamine. Onto the material for the active-substance carrier layer 2 that is initially coated over its entire surface with active-substance-containing polymer matrix 3 is guided a punching tool that comprises a hollow cylinder and a cutting blade arranged to the outside of the hollow cylinder and concentrically and directly adjoining it. In this process, the hollow cylinder is first pressed onto the coated active-substance carrier layer 2, causing a reduction in the thickness of coating with the active-substance-containing polymer matrix, with the result that a circumferential zone 4 having a reduced thickness of coating with the active-substance-containing polymer matrix is obtained. The cutting blade is then used to punch out the active substance carrier layer 2 outside the circumferential zone 4 having a reduced thickness of coating, which is applied onto a protective layer 5 furnished with a silicone coating, said protective layer completely covering the active-substance-containing polymer matrix 3 and projecting beyond the edge. In this process, the active-substance-containing polymer matrix 3 can be completely or largely completely removed in the area of the hollow cylinder, depending on the contact pressure of the hollow cylinder and the softness of the active-substance-containing polymer matrix 3. This affords an intermediate product 10, the layer structure of which is shown in FIG. 1.

On the flat side of the intermediate product 10 facing the active-substance-containing polymer matrix 3, the entire surface of the active-substance carrier layer 2 is covered by an adhesive carrier layer 6, which is bonded directly to the active-substance carrier layer 2 by means of an active-substance-free pressure-sensitive adhesive 7, affording the transdermal therapeutic system 1 of the invention. In this case too, a silicone-based pressure-sensitive adhesive is used. The bonding of the adhesive carrier layer 6 to the active-substance carrier layer 2 results in the circumferential zone 4 with a reduced thickness of coating with the active-substance-containing polymer matrix being bent over all the way round, but without this affecting its ability to act as a diffusion barrier.

After production, the transdermal therapeutic system 1 thus produced is hermetically sealed inside individual aluminum-laminated polyester film packagings and subjected to storage in a climatic chamber for 6 months at 40° C. and 75% relative humidity. The transdermal therapeutic system 1 is then taken out of the packaging and investigated for possible migration of the scopolamine. In these investigations, no scopolamine was detected in the active-substance-free pressure-sensitive adhesive 7.

To apply the transdermal therapeutic system 1 of the invention, the protective layer 5 is peeled off and the remaining layer structure is placed adhesive-side down on the desired area of skin and pressure applied.

Even after detachment of the transdermal therapeutic system 1 of the invention from the skin after a period of use of 1-2 days, analyses detected no scopolamine in the active-substance-free pressure-sensitive adhesive 7 in the zone where the adhesive carrier layer 6 projects beyond the active-substance carrier layer 2.

1.-15. (canceled)

16. A transdermal therapeutic system comprising
an active-substance carrier layer having at least one active-substance-containing polymer matrix applied to the active-substance carrier layer, said matrix comprising at least one pressure-sensitive adhesive and at least one pharmacologically active substance absorbable through human or animal skin, and
an adhesive carrier layer coated as completely as possible with an active-substance-free pressure-sensitive adhesive, said adhesive carrier layer being bonded by means of the active-substance-free pressure-sensitive adhesive directly to the flat side of the active-substance carrier layer facing away from the active-substance-containing polymer matrix,
the adhesive carrier layer projecting circumferentially beyond the edge of the active-substance carrier layer,
wherein between the active-substance-containing polymer matrix and the active-substance-free pressure-sensitive adhesive there is a circumferential zone having a reduced thickness of coating with the active-substance-containing polymer matrix and/or with the active-substance-free pressure-sensitive adhesive.

17. The system as claimed in claim 16, wherein the adhesive carrier layer is coated as completely as possible with the active-substance-free pressure-sensitive adhesive and is bonded to the active-substance carrier layer, wherein the active-substance carrier layer has a circumferential edge zone having a reduced thickness of coating with the active-substance-containing polymer matrix.

18. The system as claimed in claim 17, wherein the circumferential edge zone having a reduced thickness of coating is produced or producible by guiding a punching tool onto the material for the active-substance carrier layer that is coated over its entire surface with active-substance-containing polymer matrix, said tool comprising a hollow cylinder and a cutting blade arranged to the outside of the hollow cylinder, more particularly concentrically and directly adjoining it, the hollow cylinder being first pressed onto the coated active-substance carrier layer, causing a reduction in the thickness of coating with the active-substance-containing polymer matrix, after which the cutting blade is used to punch out the active-substance carrier layer with a circumferential edge zone having a reduced thickness of coating with the active-substance-containing polymer matrix.

19. The system as claimed in claim 16, wherein the adhesive carrier layer, where it borders the active-substance carrier layer, has a circumferential zone having a reduced thickness of coating with the active-substance-free pressure-sensitive adhesive.

20. The system as claimed in claim 16, wherein, in the system, the active-substance-containing polymer matrix is applied directly to the active-substance carrier layer and the active-substance-free pressure-sensitive adhesive directly to the adhesive carrier layer, there being no further layer between the flat side of the active-substance carrier layer facing away from the active-substance-containing polymer matrix and the active-substance-free pressure-sensitive adhesive of the adhesive carrier layer.

21. The system as claimed in claim 16, wherein the polymer matrix of the active-substance-containing polymer matrix and of the active-substance-free pressure-sensitive adhesive is independently selected from acrylates, silicone pressure-sensitive adhesives, polyisobutylenes, SIS copolymers, silicone-acrylate hybrid systems, and mixtures thereof.

22. The system as claimed in claim 16, wherein the pharmacologically active substance is selected from the group consisting of α-adrenergic agonists, β-adrenergic agonists, α-adrenergic antagonists, β-adrenergic antagonists, analgesics (narcosis-inducing), analgesics (non-narcosis-inducing), androgens, anesthetics, antiallergic s, antiandrogens, antianginals, antiarrhythmics, penicillins, antidiabetics, antidementia drugs, antihistamines, antimigraine drugs, hydrogenated ergot alkaloids, calcium-channel blockers, hormones, serotonin antagonists, platelet-aggregation inhibitors, antidepressants, bronchodilators, estrogens, gestagens, vasodilators and nicotine or mixtures thereof.

23. The system as claimed in claim 16, wherein the circumferential zone of reduced coating thickness is not more than 20% of the thickness of the polymer matrix of the active-substance-containing polymer matrix and/or of the active-substance-free pressure-sensitive adhesive, in particular not more than 15%.

24. The system as claimed in claim 16, wherein the circumferential zone of reduced coating thickness has a width of 0.05 to 5.0 mm.

25. The system as claimed in claim 16, wherein the circumferential zone of reduced coating thickness is essentially unbroken.

26. The system as claimed in claim 16, wherein the coating thickness of the active-substance-containing polymer matrix and/or of the active-substance-free pressure-sensitive adhesive is 20 to 800 μm.

27. The system as claimed in claim 16, wherein a protective layer covering the entire surface of the polymer matrix of the active-substance-containing polymer matrix and of the active-substance-free pressure-sensitive adhesive is provided, wherein the protective layer on the flat side facing the active-substance-containing polymer matrix is equipped in particular with an adhesion-reducing coating.

28. A process for producing a transdermal therapeutic system as claimed in claim 16, comprising the steps of
providing an active-substance carrier layer having at least one active-substance-containing polymer matrix applied to the active-substance carrier layer, said matrix comprising at least one pressure-sensitive adhesive and at least one pharmacologically active substance absorbable through human or animal skin, and
bonding an adhesive carrier layer coated as completely as possible with an active-substance-free pressure-sensitive adhesive by means of the active-substance-free pressure-sensitive adhesive directly to the flat side of the active-substance carrier layer facing away from the active-substance-containing polymer matrix,
wherein the adhesive carrier layer projecting circumferentially beyond the edge of the active-substance carrier layer,
wherein
between the active-substance-containing polymer matrix and the active-substance-free pressure-sensitive adhesive there is a circumferential zone having a reduced thickness of coating with the active-substance-containing polymer matrix and/or with the active-substance-free pressure-sensitive adhesive, wherein the circumferential edge zone with a reduced thickness of coating being produced by guiding a punching tool onto the material for the active-substance carrier layer that is coated over its entire surface with active-substance-containing polymer matrix, said tool comprising a hollow cylinder and a cutting blade arranged to the outside of the hollow cylinder, more particularly concentrically and directly adjoining it, the hollow cylinder being first pressed onto the coated active-substance carrier layer, causing a reduction in the thickness of coating with the active-substance-containing polymer matrix, after which the cutting blade is used to punch out the active-substance carrier layer with a circumferential edge zone having a reduced thickness of coating with the active-substance-containing polymer matrix.

29. A method comprising utilizing the transdermal therapeutic system as claimed in claim 16 for the treatment of the animal or human body, wherein the treatment is selected from hypogonadism, hormone replacement therapy, Alzheimer's, of Parkinson's, of multiple sclerosis, bipolar disorders, muscle tension, severe pain, high blood pressure or for contraception.

30. A kit comprising at least one transdermal therapeutic system as claimed in claim 16 in an outer packaging and instructions for use.
